(19) United States
(12) Patent Application Publication
(10) Pub. No.: US 2003/0149255 A1
(43) Pub. Date: Aug. 7, 2003

(54) Bird diagnostics and treatments

(76) Inventors: Vanessa Lopes, St Paul, MN (US); Kakambi V Nagaraja, St Paul, MN (US)

Correspondence Address: FISH & RICHARDSON P.C., 3300 DAIN RASCHER PLAZA, 60 SOUTH SIXTH STREET, MINNEAPOLIS, MN, 55402, US

(21) Appl. No.: 10/148,139

(22) PCT Filed: Oct. 9, 2001
(86) PCT No.: PCT/US01/31569

(51) Int. Cl. C07H 21/02; C07H 21/04

(52) U.S. Cl. 536/23.1

(57) ABSTRACT

The invention relates to methods and materials for protecting birds from infection with ORT as well as for detecting ORT infection in birds. Specifically, the invention provides ORT antigens, anti-ORT antibodies, and mutant ORT organism, as well as methods and materials for detecting and preventing ORT infections.

### TECHNICAL FIELD

The invention relates to methods and materials for protecting birds from infection by Ornithobacterium rhinotracheale (ORT) as well as for detecting ORT infection in birds.

### BACKGROUND

Ornithobacterium rhinotracheale (ORT) is a pleomorphic, rod-shaped gram-negative bacterium associated with respiratory disease in poultry. The poultry industry has suffered significant financial losses due to the drop in egg production, growth suppression, mortality and condemnation of carcasses in flocks infected with this organism. The clinical signs in turkeys and chickens infected with ORT include coughing, nasal discharge, arthritis and prostration. The gross lesions in turkeys due to ORT infection include edema consolidating in the lungs, sinusitis, pericarditis, hepatomegally and airsacculitis. In chickens infected with ORT, signs of pneumonia and airsacculitis has been observed. The clinical signs and lesions caused by ORT infection are very similar to those caused by other respiratory infectious agents. Mortality rates due to pneumonia in birds infected with ORT can be as high as 15 percent (%).

ORT was first isolated in 1991 from broilers in Germany with respiratory disease. This organism was subsequently detected in chickens and turkeys in the United States, South Africa, France, Netherlands, Hungary and Israel and, more recently, in Canada and Austria. The role of ORT in respiratory disease in turkeys and chickens has been demonstrated. In addition to turkeys and chickens, ORT has also been isolated from ducks, partridges, rooks and guinea fowl. Twelve different ORT serotypes designated A through L have been reported. Serotype A is the most prevalent in chickens. The ORT serotypes in turkeys, however, are more heterogeneously distributed.

### SUMMARY

The invention provides methods and materials for identifying birds exposed to or infected with ORT as well as methods and materials for protecting birds from infection with ORT. The materials and methods of the invention that can be used to detect ORT or ORT infections in birds provide superior sensitivity and the ability to detect ORT infections early relative to existing assays. Methods and materials of the invention that are effective for protecting birds from ORT infection can be readily administered to a large number of birds.

In one aspect, the invention provides a composition including an isolated mutant ORT organism. In another aspect of the invention, there is provided a vaccine that includes an effective amount of a mutant ORT organism. The isolated mutant ORT organism can be attenuated. In addition, the isolated mutant ORT organism is a temperature sensitive mutant ORT organism having a permissive temperature for growth and a non-permissive temperature for growth. A permissive temperature includes 31° C., while a non-permissive temperature includes 41° C. Representative permissive temperatures include from about 29° C. to about 33° C. Representative non-permissive temperature includes below 29° C. and above 33° C. The mutant temperature sensitive ORT organism can be alive. A mutant temperature sensitive ORT organism can include the distinguishing characteristics of the organism assigned ATCC Accession number ______, and can be designated VL mORT 108a-10.6. Typically, the composition, after administration, protects a bird from an ORT infection. Further, the composition can be effective for lowering the risk of an ORT infection in a bird.

In yet another aspect, the invention provides methods for protecting a bird from an ORT infection, the method comprising administering to the bird a composition comprising an effective amount of an isolated mutant ORT organism. Further provided by the invention are methods for reducing the risk of an ORT infection in a bird, comprising administering to the bird a composition comprising an isolated mutant ORT organism. The composition can be applied to an eye or a nostril of the bird, or can be supplied in the drinking water. Representative examples of birds are turkeys a chickens.

In another aspect of the invention, there is provided an inoculated bird, wherein the bird comprises anti-ORT antibodies due to inoculation with an isolated mutant ORT organism. The inoculated bird can be a turkey or a chicken. The invention further provides body parts such as a meat portion of such an inoculated bird.

In yet another aspect, the invention provides methods for identifying a bird that is or was infected with ORT, the method comprising: a) contacting a biological sample from the bird with an ORT antigen under conditions wherein the ORT antigen specifically binds to an anti-ORT antibody, if present in the biological sample, to form an antibody-antigen complex; and b) detecting the presence or absence of the antibody-antigen complex, the presence of the antibody-antigen complex indicating that the bird is or was infected with ORT. Representative birds are turkeys and chickens. The ORT antigen can comprise an ORT OMP preparation such as an ORT serotype A OMP preparation, an ORT serotype C OMP preparation, an ORT serotype E OMP preparation, or an ORT serotype I OMP preparation. The ORT antigen can comprise a 70% pure ORT polypeptide. Such an ORT antigen can be immobilized on a solid support. Typical solid supports include a dipstick, a microtiter plate, a bead, an affinity column, and an immunoblot membrane. A representative biological sample includes serum. The detecting step of such a method can include performing an enzyme-linked immunoassay, a radioimmunoassay, an immunoprecipitation, or an immunoblot assay. In particular, the detecting step can include: contacting the antibody-antigen complex with an indicator molecule that selectively binds to the anti-ORT antibody; and detecting the presence of the indicator molecule.

In another aspect, the invention also provides methods for detecting an ORT infection in a bird, the method comprising: a) contacting a biological sample from the bird with an anti-ORT antibody under conditions wherein the anti-ORT antibody specifically binds to an ORT antigen, if present in the biological sample, to form an antibody-antigen complex; and b) determining the presence or absence of the antibody-antigen complex, the presence of the antibody-antigen complex indicating that the bird has the infection. The ORT antigen can include a portion of an ORT organism. In particular, the ORT antigen can be one or more ORT OMPs. The anti-ORT antibody can be immobilized on a solid support. Typically, the anti-ORT antibody has specific binding affinity for an OMP polypeptide. Generally, the biological sample comprises a tracheal swab.

It is another aspect of the invention to provide a composition comprising a substantially pure ORT antigen. It is an aspect of the invention to provide a composition consisting essentially of an ORT OMP preparation. In addition, it is an aspect of the invention to provide an ORT antigen and an indicator molecule. It is another aspect of the invention to provide a composition comprising an anti-ORT antibody and an indicator molecule. Such compositions of the invention can further comprise one or more antigens or antibodies for detecting a plurality of avian infections. Representative avian infections can be caused by an organism selected from the group consisting of Salmonella spp., Bordetella avian, avian pneumovirus, avian encephalitis virus, avian influenza, avian leukosis, fowl pox, infectious bronchitis virus, infectious bursal disease virus, Newcastle dsease virus and reovirus. Generally, the indicator molecule selectively binds to anti-ORT antibodies produced by the bird species from which the biological sample is obtained. The ORT antigen can be immobilized on a solid substrate such as a dipstick, a microtiter plate, a bead, an affinity column, an immunoblot membrane, and an immunoblot paper.

Unless otherwise defined, all technical and scientific terms used herein have the same meaning as commonly understood by one of ordinary skill in the art to which this invention pertains. Although methods and materials similar or equivalent to those described herein can be used in the practice or testing of the invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 depicts the detection of ORT infection using whole cell antigen in a SPAT. Sera from birds infected with serotypes A, C, E and I were tested with the polyvalent whole cell antigen. The average percentage of positive birds from all 4 serotypes is plotted.

FIG. 2 depicts the detection of ORT infection in an ELISA using outer membrane proteins of ORT. Turkeys were infected with ORT serotypes A, C, E, and I via oculonasal. The ELISA results were read at 405 nm and are expressed in number of positive birds. The average percentage of positive birds from all 4 serotypes is plotted.

FIG. 3 depicts the percentage of positive birds positive for ORT antibodies in vaccinated and non-vaccinated turkeys with different concentrations of Ts-ORT strain of ORT in drinking water or oculonasal instillation. All groups of birds were vaccinated at 4 wks. (a) non-vaccinated; (b) drinking water vaccination (10⁷ CFU/ml); (c) drinking water vaccination (10⁸ CFU/ml); (d) oculonasal vaccination (10⁷ CFU/ml); (e) oculonasal vaccination (10⁸ CFU/ml); (f) oculonasal vaccination (10⁹ CFU/ml).

FIG. 4 depicts the antibody titers to ORT in vaccinated and non-vaccinated turkeys measured by OMP-ELISA. The dashed line indicates the cut-off value for a positive serum.

### DETAILED DESCRIPTION

The invention provides methods and materials related to detecting ORT infections in wild and domesticated birds such as turkeys, chickens, quails, ducks, partridges, rooks, pheasants, guinea fowls, and geese. The materials and methods described herein can be used to detect infection by any ORT serotype. In addition, the invention provides materials and methods related to protecting birds from ORT infection. For example, the invention provides temperature sensitive mutant ORT strains and methods for their use as vaccines to protect birds from ORT infections.

Production of ORT Antigens

The invention provides methods of detecting an ORT infection in a bird. In one embodiment, an ORT antigen can be used to detect an anti-ORT antibody in a biological sample collected from a bird. A bird that contains anti-ORT antibodies has been exposed to or infected with ORT. Different ORT antigens can be used in combination to detect anti-ORT antibodies in birds due to infection with different ORT serotypes (e.g., serotype A, serotype B, serotype C, serotype D, serotype E, serotype F, serotype G, serotype H, serotype I, serotype J, serotype K, or serotype L, serotype M, serotype N, serotype 0, and others).

ORT antigens that are particularly useful in methods of the invention include, without limitation, one or more purified ORT outer membrane proteins (OMPs). Thus, ORT antigens can be a single or a combination of purified OMPs (or a fragment or fragments thereof) from one ORT serotype, or a single or a combination of purified OMPs or fragments thereof from multiple ORT serotypes. The term “purified” as used herein with reference to one or more polypeptides (e.g., OMPs) means that the polypeptides have been at least partially removed from their natural environment, i.e., the polypeptides have been at least partially separated from cellular components that naturally accompany them. Typically, a polypeptide is purified when it is at least 60% (e.g, 70%, 80%, 90%, 95%, or 99%), by weight, free from, for example, non-OMPs and naturally occurring organic molecules that are associated with the polypeptide. A polypeptide suitable for use as an ORT antigen is typically a chain of at least five amino acids that contains an epitope recognized by an anti-ORT antibody.

ORT antigens (e.g., ORT OMPs) that are useful for detecting ORT infections can be obtained from a number of sources, including, without limitation, ORT cells and biological samples collected from ORT-infected birds. For example, an ORT antigen can be obtained by purifying OMPs from an ORT culture using methods such as those described herein. In addition, an ORT antigen, such as OMPs from ORT, can be obtained from a blood sample collected from an ORT infected bird. Recombinant techniques also can be used to obtain an ORT antigen. For example, an ORT antigen also can be produced by ligating nucleic acid sequences encoding one or more ORT polypeptides (e.g., OMPs) into a construct such as an expression vector, and introducing the construct into a bacterial or eukaryotic host cell by routine methods such as electroporation, calcium phosphate, or other suitable method. The cells can be cultured under conditions appropriate for expression of the nucleic acid sequences, and the ORT polypeptides can be purified. Expression vectors (e.g., glutathione S-transferase (GST)- and His6×tag-containing constructs) that aid in purification of the fusion protein product can be used and are commercially available. Further, ORT polypeptides, e.g., OMPs, to be used as ORT antigens, can be chemically synthesized using standard techniques. See, Muir & Kent, Curr. Opin. Biotechnol., 4(4): 420-427 (1993), for a review of polypeptide synthesis techniques. An immunoaffinity column in which anti-ORT antibodies are immobilized on a suitable column media can be used to purify, for example, chemically synthesized ORT polypeptides or ORT polypeptides made using a heterologous expression system.

Production of Anti-ORT Antibodies

The invention provides methods and materials for detecting an ORT infection by detecting an ORT antigen in a biological sample collected from a bird. Anti-ORT antibodies having specific binding affinity for an ORT antigen, e.g., ORT OMPs, can be used to detect an ORT antigen in a biological sample collected from a bird. The term “anti-ORT antibodies” as used herein refers to antibodies that have specific binding affinity for an ORT antigen. Suitable anti-ORT antibodies can have similar binding affinities for ORT antigens from multiple ORT serotypes, or anti-ORT antibodies can have different binding affinities for ORT antigens from specific ORT serotypes. Anti-ORT antibodies include, without limitation, intact molecules as well as fragments thereof that are capable of binding to an ORT antigen. Thus, the terms “antibody” and “antibodies” include polyclonal antibodies, monoclonal antibodies, humanized or chimeric antibodies, single chain Fv antibody fragments, Fab fragments, and F(ab)₂ fragments. The term “epitope” refers to an antigenic determinant on an antigen to which the paratope of an antibody binds. Epitopes usually consist of chemically active surface groupings of molecules such as amino acids or sugar side chains, and typically have specific three-dimensional structural characteristics, as well as specific charge characteristics. Epitopes generally include at least five contiguous amino acid residues.

Methods of generating anti-ORT antibodies are known in the art. For example, an ORT antigen can be produced as described above (e.g., by purifying a native ORT polypeptide, by expressing an ORT polypeptide using an expression construct, or by chemically synthesizing an ORT polypeptide), and then used to immunize an animal. Various host animals including, for example, rabbits, chickens, mice, guinea pigs, and rats, can be immunized by injection of one or more ORT polypeptides. Depending on the host species, adjuvants can be used to increase the immunological response. In particular, monoclonal antibodies can be obtained by any technique that provides for the production of antibody molecules by continuous cell lines in culture such as described by Kohler et al., Nature, 256:495 (1975), the human B-cell hybridoma technique of Kosbor et al., Immunology Today, 4:72 (1983) and/or Cole et al., Proc. Natl. Acad. Sci. USA, 80:2026 (1983), and the EBV-hybridoma technique of Cole et al., “Monoclonal Antibodies and Cancer Therapy”, Alan R. Liss, Inc. pp. 77-96 (1983). Antibodies can be of any immunoglobulin class including IgM, IgG, IgE, IgA, IgD, and any subclass thereof. A hybridoma producing monoclonal antibodies of the invention can be cultivated in vitro or in vivo.

A chimeric antibody is a molecule in which different portions of the antibody are derived from different animal species, such as an antibody having a variable region derived from a murine monoclonal antibody and a constant region derived from a human immunoglobulin. Chimeric antibodies can be produced through standard techniques. In addition, antibody fragments can be generated by known techniques. For example, antibody fragments such as F(ab′)₂ fragments can be produced by pepsin digestion of an antibody molecule. Fab fragments can be generated by deducing the disulfide bridges of F(ab′)₂ fragments. Single chain Fv antibody fragments can be produced by linking the heavy and light chain fragments of a Fv region via an amino acid bridge (e.g., 15 to 18 amino acids). See, for example, U.S. Pat. No. 4,946,778.

Once produced, antibodies or fragments thereof can be tested for the ability to bind an ORT antigen by standard immunoassay methods including, for example, emzyme-linked immunoassays (ELISA), radioimmunoassays (RIA), immunoprecipitation, fluorescence assays, chemiluminescent assays, immunoblot assays, or particulate-based assays. See, Short Protocols in Molecular Biology, Chapter 11, Ausubel et al., (eds.), Green Publishing Associates and John Wiley & Sons (1992).

Detecting ORT Infections

The invention provides methods and materials for identifying birds that are or were infected with ORT. Thus, ORT antigens can be used to detect anti-ORT antibodies, thereby identifying birds that are or were infected with ORT. Likewise, anti-ORT antibodies can be used to detect ORT antigens, thereby identifying birds having an ORT infection.

As used herein, infection is understood to mean that ORT bacteria are present and multiplying in the bird. A bird that was infected with ORT refers to a bird that recovered from an ORT infection and is no longer experiencing clinical symptoms. Birds that were or are infected with ORT can be identified using a biological sample collected. Such biological samples can be collected at any time (e.g., 3 days, 7 days, 10 days, 2 weeks, or 6 weeks post-infection). Biological samples include, for example, whole blood, plasma, serum, feces, tissues, and any other material collected from a bird. Generally, anti-ORT antibodies can be detected in birds at 1, 2, 3, 4, 5, 6, 7, and 8 weeks post-infection, while ORT antigens can be detected in birds at 2, 3, 5, 6, 8, 10, 12 and 14 days post-infection.

In one embodiment of the invention, an ORT antigen can be immobilized on a solid substrate such as a dipstick, a microtiter plate, particles (e.g., beads), an affinity column, and an immunoblot membrane and used to detect anti-ORT antibodies in a biological sample from a bird. See, U.S. Pat. Nos. 5,143,825, 5,374,530, 4,908,305, and 5,498,551 for exemplary descriptions of solid substrates and methods for their use. For example, an ORT polypeptide can be immobilized on a solid substrate, such as a 96-well plate, using known techniques, then contacted with the biological sample under conditions such that anti-ORT antibodies, if present in the biological sample, bind to the immobilized antigen to form an antibody-antigen complex. Suitable conditions to form an antibody-antigen complex include incubation in an appropriate buffer (e.g., sodium carbonate buffer, pH 9.5) at room temperature from about at least 10 minutes to about 10 hours (e.g., from about 1 to about 2.5 hours). Thereafter, unbound material can be washed away, and an antibody-antigen complex can be detected.

Alternatively, an anti-ORT antibody can be immobilized on a solid substrate using known methods and used to detect an ORT antigen in a biological sample collected from a bird. The immobilized anti-ORT antibody can be contacted with a biological sample under conditions such that an antigen-antibody complex is formed if ORT antigens are present in the biological sample. In some embodiments, antibody-antigen complexes are formed in solution. Such complexes can be detected using routine immunoprecipitation procedures. See, e.g., Short Protocols in Molecular Biology, Chapter 10, Section VI, Ausubel et al., (eds.), Green Publishing Associates and John Wiley & Sons (1992).

The presence of antibody-antigen complexes can indicate that the bird was or is infected with ORT. In general, antibody-antigen complexes can be detected using an indicator molecule having specific binding affinity for either the antigen or the antibody of an antibody-antigen complex or the antibody-antigen complex itself. As used herein, an indicator molecule is any molecule that allows the presence of a given antigen, antibody, or antibody-antigen complex to be detected, either with the naked eye or an appropriate instrument. The indicator molecule can be an antibody having specific binding affinity for antibodies from the bird species from which the biological sample was obtained, e.g., an anti-turkey IgG antibody (Rockland Immunochemicals, Gilbertsville, Pa.) or can be an antibody having specific binding affinity for antibodies from the species from which the anti-ORT antibodies were generated (e.g., an anti-goat IgG antibody).

Indicator molecules can be detected either directly or indirectly by standard methodologies. See, e.g., Current Protocols in Immunology, Chapters 2 and 8, Coligan et al., (eds.), John Wiley & Sons (1996). For direct detection, the indicator molecule or the ORT antigen can be labeled with a radioisotope, fluorochrome, other non-radioactive label, or any other suitable chromophore. For indirect detection methods, enzymes such as horseradish peroxidase (HRP) and alkaline phosphatase (AP) can be attached to the indicator molecule, and the presence of the antibody-antigen complex can be detected using standard assays for HRP or AP. Alternatively, the indicator molecule can be attached to avidin or streptavidin, and the presence of the antibody-antigen complex can be detected with biotin conjugated to, for example, a fluorochrome. Thus, assay formats for detecting antibody-antigen complexes can include enzyme-linked immunoassays (ELISA) (e.g., a competitive ELISA, radioimmunoassays (RIA), fluorescence assays, chemiluminescent assays, immunoblot assays (Western blots), particulate-based assays, and other known techniques.

ORT antigens and/or anti-ORT antibodies that are effective for identifying birds that were or are infected with ORT as described herein can be combined with packaging material and sold as a kit. Components and methods for producing such kits are well known. The kits can combine one or more ORT antigens such as OMPs from different serotypes. Instructions describing how an ORT antigen or anti-ORT antibody is effective for identifying birds that are or were infected with ORT can be included in such kits.

In addition, a kit can include antibodies, antigens, indicator molecules, and/or useful agents for detecting other avian diseases. For example, the kits described herein can be used to determine if a bird has an ORT infection, another bacterial infection (e.g., Salmonella spp., or Bordetella avian), a mycoplasma infection (e.g., Mycoplasma gallisepticum or Mycoplasma synoviae), or a viral infection such as a viral infection caused by avian pneumovirus (APV), avian encephalitis virus (AEV), avian influenzavirus, avian leucosis virus (ALV), fowl pox, infectious bronchitis virus (IBV), infection bursal disease virus (IBD), Newcastle disease virus (NDV, also known as paramyxovirus-1, PMV-1), PMV-2, PMV-3, or a reovirus. For example, a kit of the invention can include a solid substrate onto which ORT antigens and other suitable antigens or reagents capable of detecting other avian disease-causing organisms or viruses have been immobilized in different, discrete regions. Appropriate immunoassays can be performed using such a kit and a biological sample as described above.

Mutant ORT Organisms

The compositions described herein provide an effective way for preventing, ameliorating, lowering the risk of, lowering the occurrence of, and/or spread of ORT infections in birds. In particular, the compositions described herein are useful for vaccinating birds living in flocks or other types of close living arrangements where an ORT infection can rapidly spread from bird to bird. Any ORT organism can be used to prepare a mutant ORT organism. Mutant ORT organisms can be developed to correspond to one or more ORT serotypes. ORT serotypes include serotype A, serotype B, serotype C, serotype D, serotype E, serotype F, serotype G, serotype H, serotype I, serotype J, serotype K, and serotype L, serotype M, serotype N, serotype O and others. Combining mutant ORT organisms from more than one serotype can enhance the immunogenic response in a bird.

The term “mutant ORT organism” as used herein is an ORT organism that (1) is not naturally occurring in nature and (2) contains a genetic modifications. Genetic modifications include insertions, deletions, translocations, transversions, transitions, and combinations thereof. Mutant ORT organisms can be generated using any known method for mutagenizing bacteria. In particular, chemical mutagenesis as well as other forms of mutagenesis (e.g., ultraviolet light, and site directed mutagenesis) can be used to produce mutant ORT organisms. By way of example, wild-type ORT strains can be isolated as described (Sprenger et al., 1998, Avian Dis., 42:154-61) and treated with a chemical mutagen such as N-methyl-N′-nitro-N-nitrosoguanidine (MNNG). ORT organisms can be exposed to various concentrations of MNNG, for example, 500, 1000, 1500, 2000 μg/ml amounts of mutagen for at least 48 hours. Multiple rounds of subculturing (e.g., 2, 3, 4, 5, 6, 7, 8, 9 or 10 rounds) can be performed. Stable mutants that do not undergo genetic reversion can then be identified.

Mutant organisms can be characterized and isolated using methods routine in the art (see, for example, Chatfield et al., 1989, Vaccine, 7: 495-8 as well as methods described herein). Mutant ORT organisms typically grow slower and produce smaller colonies relative to a wild-type parent ORT organism. It is generally desirable that mutant ORT organisms resemble the parent strain morphologically as well as in biochemical characteristics that can be determined by an APZyme assay. Typically, non-mutagenized parent strains of ORT grow optimally at 41° C., but can grow at a variety of temperatures ranging from about 29° C. to about 45° C. Temperature sensitive (Ts) mutant ORT organisms generally grow under a limited temperature range as compared to the temperature range for a wild-type parent strain. Based on the temperature gradient that is present in the respiratory system of birds, mutant organisms that are temperature sensitive can colonize the upper respiratory tract and stimulate local immunity by eliciting production of IgA. The higher temperature (e.g., 41° C.) of the lower respiratory tract and other internal organs, however, is non-permissive for the temperature sensitive mutants, thereby avoiding the development of severe lung lesions due to systemic entry of the organism. Thus, suitable temperature sensitive mutant ORT organisms can grow at temperatures from about 29° C. to about 33° C. (e.g., 31° C.). In particular, a Ts-ORT organism can be isolated using the methods described in U.S. Pat. No. 6,077,516.

A mutant ORT organism is sufficiently attenuated and useful as a vaccine when a dosage of about 10⁵ CFU elicits an immunological response in a bird but does not cause the bird to develop severe clinical signs of an ORT infection. A useful mutant ORT organism includes ORT vaccine VL mORT 108a-10.6. The mutant ORT organism, VL mORT 108a-10.6, can be used as a vaccine that is safe and immunogenic in turkeys. VL mORT 108a-10.6 was deposited with the American Type Culture Collection (ATCC, 10801 University Blvd., Manassas, Va., 20110-2209) on ______ and received ATCC Accession number . A bird that has received an effective dosage is a vaccinated bird or an inoculated bird, i.e., the bird contains anti-ORT antibodies due to inoculation with an isolated mutant ORT organism. Inoculated birds become seropositive for anti-ORT antibodies and resistant to infection by a virulent ORT.

Methods for detecting an immunological response in a bird are known and illustrative examples are provided herein. Vaccinated birds that are subsequently exposed to a virulent ORT organism can still pass slaughter inspections and continue to market. Methods and rating systems for passing or condemning birds destined for slaughter are known. Virulent ORT organisms are those ORT organisms that infect a bird causing clinical symptoms of an ORT infection (e.g., coughing, nasal discharge, arthritis, and prostration).

Effective dosages can be determined experimentally. Effective doses can be at least about 10⁵ CFU/bird. Dosages may vary according to the type, size, age, and health of the bird to be vaccinated. For example, an effective amount for a two-week-old turkey poult may include an ORT vaccine dosage of about 10⁵, 10⁶, or 10⁷ CFU/bird. Older turkeys may require larger dosages (e.g., greater than 10⁵, 10⁶, 10⁷, 10⁸, 10⁹, or 10¹⁰ CFU/bird). The vaccination may include a single inoculation or multiple inoculations. Other dosage schedules and amounts including vaccine booster dosages can be used.

The vaccination schedule may depend upon the type of bird and the purpose for which the bird is being kept. For example, it may be preferable to inoculate meat-producing birds at a young age, perhaps as newborns or hatchlings, or when the birds are only a few weeks old. Alternatively, it may be useful to vaccinate egg-producing birds at other times, e.g., shortly before they are about to lay (perhaps with a vaccine booster dosage) so that maternal antibodies may be transmitted to the young. Of course, it may also be useful to inoculate egg-laying birds at an early age to prevent ORT infection in the egg-laying flock.

An effective dosage can be given to a bird using any known method including direct application intranasally, intraocularly, and/or as a subcutaneous or intramuscular injection. In addition, an effective dosage can be given to a representative sample or subset of a flock. In this case, the inoculated birds can be allowed to commingle with the rest of the flock such that other birds are passively inoculated. Inoculating a subset of the flock can create a rolling or sequential vaccination as the mutant ORT organism is passed from bird to bird. The number of vaccinated birds in the flock can increase as the directly vaccinated birds interact with the rest of the flock. In the end, a majority or all of the birds can become vaccinated.

Alternatively, an effective dosage of a vaccine may be given directly to each member of a flock, or the dosage can be applied to the food and/or water supply of a flock. Most, if not all, members of a flock can become vaccinated at about the same time when inoculation is via the food or water supply. Dosages administered through the food or water supply can be easily computed by multiplying the amount a single bird eats or drinks per day by the number of birds to be inoculated to compute the unit of food or water consumed per day per bird. Then, the unit of food or water consumed per day is used to compute the vaccine dosage needed to dissolve in that unit of food or water so 4s to deliver at least 10⁵ CFU/bird.

Compositions containing a mutant ORT organism also have uses other than as a vaccine. For example, such compositions can be used to induce a bird to raise antibodies to ORT to be used in diagnostic tests for identifying one or more ORT isolates. Further, a mutant ORT organism can be used in a diagnostic assay for detecting the presence of anti-ORT antibodies in the sera of a bird.

Compositions Containing Mutant ORT Organisms

It is to be understood that mutant ORT organisms themselves and/or compositions that include mutant ORT organisms can include other components conventional to the art such as an adjuvant, sterile water, pharmaceutically acceptable carriers, vaccine carriers, and buffers that are useful for maintaining the viability of the mutant ORT organism. A composition can be in an effective amount of a mutant ORT organism appropriate for a particular type of bird, administration route and schedule.

A composition containing a mutant ORT organism can contain other mutant, attenuated or inactivated bacterial or viral strains, microorganisms, and antigens, which, for example, can protect the inoculated birds against other avian diseases. Mutant ORT organisms may be combined with different vaccines or preventative methods directed to other avian diseases so as to produce birds that are relatively pathogen free, healthier, and/or resistant to multiple avian diseases. Other avian diseases include Salmonella spp. infections, Bordetella avium infections, avian influenza, avian pneumovirus infection, New Castle Disease, Mycoplasma spp. infections, and Pasteurella multocida infections. Methods for producing such multi-effect vaccines are known. Conveniently, a mutant ORT organism may be provided in a pre-packaged form in quantities sufficient for a protective dose for a single bird or for a pre-specified number of birds in, for example, sealed ampoules, capsules, or cartridges.

The invention will be further described in the following examples, which do not limit the scope of the invention described in the claims.

### EXAMPLES

### Example 1

### Bacterial Strains

Four strains of ORT from the University of Minnesota, Department of Veterinary PathoBiology laboratory collection were used in this study. ORT-UMN 108 (serotype A), ORT-UMN 32 (serotype C), ORT-UMN 87 (serotype E), and ORT-UMN 18 (serotype I). The ORT serotypes (A, C, E, and I) were grown in 5% sheep blood agar (SBA) plates containing 10 μg/ml gentamicin (Signa-Aldridch Co., St. Louis, Mo.), as previously described (Back, Proc. Turkey ORT Symposium, 1996, pp 29-31). The Ts-ORT strain was grown on SBA plates at 31° C. under the conditions described. Bacterial cells were aseptically harvested and diluted in sterile phosphate-buffered saline (PBS; pH 7.4) to 10⁷, 10⁸ and 10⁹ colony-forming units per milliliter (CFU/ml).

### Example 2

### Extraction of OMPs

OMPs were extracted by a standard procedure (Todhunter et al., 1991, Vet. Immunol. Immunopathol. 28:107-15) with slight modifications. The OMPs from each ORT serotype were extracted from a 24-hr culture. Cells from each serotype were separately harvested, resusupended in 0.85% NaCl, and disrupted by probe sonication. The intact cells and insoluble debris were removed by centrifugation at 5000×g for 20 min. The supernatant was harvested and centrifuged at 100,000×g for 60 min. The high-speed centrifugation allowed OMPs to pellet at the bottom of the tube. The pellet was resuspended in 10 ml 25 mM Tris-HCl (Bio-Rad Laboratories, Hercules, Calif.) and treated with 2% sodium N-lauroylsarcosine (Sigma-Aldrich Co.). The insoluble proteins were again pelleted by centrifugation at 100,000×g for 60 min. The pellet was resuspended in 10 ml 25 mM Tris-HCl and stored at -20° C.

### Example 3

### Sodium Dodecyl Sulfate-Polyacrylaminde Gel Electrophoresis (SDS-PAGE), and Western Blot

The extracted OMPs were separated by SDS-PAGE as described (Todhunter et al., 1991) for protein profile analysis. The OMPs of each serotype (2 mg/ml) were diluted 1:2 in SDS buffer, incubated at 65° C. and then loaded onto the gel. The SDS-PAGE was performed with 4% stacking gel and 12% separating gel and run at 100 volts for 2 hours.

Western blot analysis of ORT OMPs was performed by electrophoretically transferring according to the procedure described (Todhunter et al., 1991). The OMPs were transferred to a nitrocellulose membrane. Four blots, each one containing OMPs from ORT serotypes A, C, E and I, were blotted with antiserum against ORT serotypes A, C, E and I, respectively, and compared for antigenic analysis across OMPs.

OMP of four different serotypes of ORT (A, C, E, and I) were examined by SDS-PAGE and analyzed. The SDS-PAGE revealed a high similarity of protein profiles among most of the OMPs of different serotypes of ORT. There were very few differences in the protein profiles between the serotypes examined.

The hyperimmune sera of different ORT serotypes were capable of detecting OMP antigens of distinct serotypes of ORT. The Western blot analysis of these proteins detected the existence of several common protein bands among different serotypes of ORT, suggesting that OMP from a unique ORT serotype can be used as an antigen for detection of different ORT serotypes by immunodiagnostic techniques.

### Example 4

### ELISA and Serum Plate Agglutination Test (SPAT)

Thirty turkey poults known to be free of antibodies to ORT as tested by serum plate agglutination test (SPAT) were raised in isolation. They were equally divided into 5 groups. At 3 wks of age, birds in groups 1, 2, 3 and 4 were exposed by oculonasal routes with 0.5 ml of ORT culture serotypes A, C, E and I, respectively, containing 2.3×10⁹ CFU/ml. Birds in group 5 were kept as uninoculated controls. The birds were kept in isolation until 8 wks post-infection. At weekly intervals, blood samples from ORT-exposed and non-exposed birds were collected and examined for the presence of antibodies against ORT by ELISA and SPAT. The results of ELISA were compared with the results of SPAT.

OMPs from ORT serotype A were used as an antigen in ELISA. ELISA was performed as described previously (Heckert et al., 1994, Avian Dis., 38 :694-700) with slight modifications. For standardization of indirect ELISA for ORT, 96-well-polystyrene plates (Nunc-immuno Module; Life Technologies, Burlington, Ontario, CANADA) were coated for 4 hrs at room temperature with different dilutions of the OMPs of ORT serotype A, and then the different diluted OMP samples were evaluated by using hyperimmune sera raised in turkeys against ORT. Horseradish peroxidase-labeled antibodies to turkey IgG (Kirkergaard & Perry Laboratories, Gaithersburg, Md.) and 2,2′-azino-di (3-ethyl-benzthiazoline-6-sulfonate) (ABTS Microwell Peroxidase Substrate; Kirkergaard & Perry Laboratories) were used as conjugate and substrate, respectively. The optical density (OD) values were read at a wavelength of 405 nm. The ELISA cut-off point was determined by using sera from 40 known negative turkeys. This point was calculated as the average OD of negative samples plus twice the standard deviation. ORT OMP-coated plates were also examined for cross reactivity to the antisera from other gram-negative bacteria such as Salmonella serogroups B and D and E. coli serogroups 01a, 02a, and 078.

SPAT using whole ORT cell antigen was performed following the procedure described (Back et al., 1998, J. Vet. Diag. Invest., 10:84-6). Polyvalent whole cell antigen for SPAT was prepared by mixing equal volumes of whole cell antigen from serotypes A, C, E, and I. The test was standardized using known positive and negative serum from ORT.

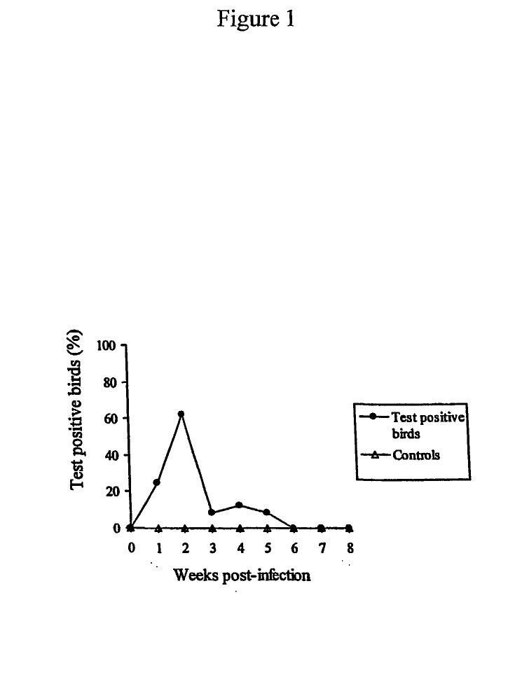

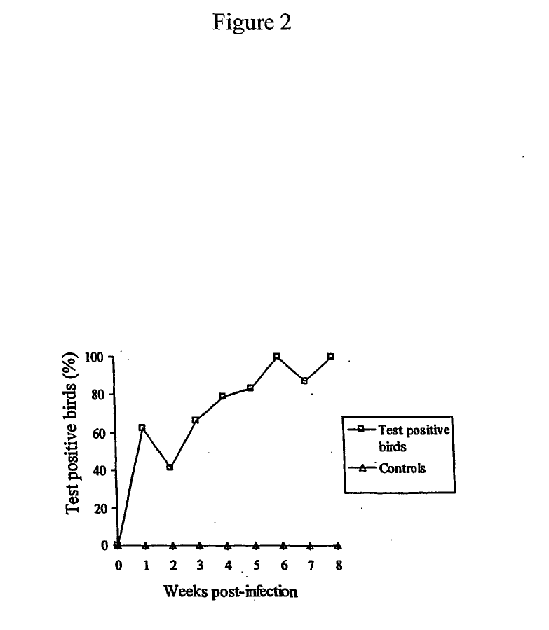

The results are shown in Table 1 and plotted in FIGS. 1 and 2. The SPAT with whole cell antigen detected specific antibodies of ORT in 65% of birds during the first 2 wks of infection. The ELISA containing OMP as an antigen was able to detect specific antibodies against ORT in up to 100% of the infected birds for 8 wks post-infection. The OMP of ORT did not react with sera from Salmonella (serogroups B and D) and E. coli 01A, 02A, and 078, indicating the absence of cross-reactivity with these gram-negative organisms.

The results suggest that after the initial stage of the infection, there was a decline in the detection of antibodies by the SPAT, showing a decrease in the sensitivity of the test. The initial stage of the infection has high levels of IgM antibodies, which are very efficient in agglutination with specific antigens. The ELISA results exhibited a higher sensitivity (t=4.51, p<0.0025) in the detection of specific antibodies for ORT after 2 wks post-infection compared with SPAT. In addition, the OMP or ORT in an ELISA test exhibited cross-reaction among different serotypes. ELISA with OMP or ORT can be used in serologic surveillance of ORT infection. In addition, the test can be automated to handle large numbers of samples, unlike with SPAT.

### Example 5

### Development of the Ts-ORT Strain

The wild-type strain of ORT was grown on SBA plates at 37° C. as described herein. The bacterial cells were harvested and resuspended in pre-warmed tryptic soy broth (TSB) containing 1,500 μg/ml of MNNG (Sigma-Aldrich Co.) following a modified procedure (Emery, 1989, M.Sc. Thesis, Univ. of MN). The cells were incubated with MNNG at 37° C. for 30 mins and washed with cold PBS at pH 7.4 to eliminate residual mutagen. The pelleted cells were resuspended in pre-warmed TSB and incubated at 31° C. for 40 mins. The MNNG-exposed bacteria were plated in 10-fold dilutions onto SBA plates and incubated at 31° C. The plates containing about 150 colonies were selected and replica-plated using a colony transfer pad (Schleicher & Schuell, Keene, N.H.) onto two SBA plates. One plate was incubated at 31° C., and the other at 41° C. for at least 48 hrs. The Ts-ORT colonies were selected based on colony size, slow growth at 31° C. (permissive temperature), and inhibition of growth at 41° C. (non-permissive temperature). The first-step Ts-ORT organisms were again treated with MNNG (1,500 μg/ml), plated and selected. The stability of 480 Ts-ORT colonies was assessed in vitro by culturing 20 successive passages onto SBA plates at permissive and non-permissive temperatures. One strain that showed no reversion growth at non-permissive temperature was selected and was designated VL mORT 108a-10.6 (ATCC Accession No. ______).

### Example 6

### Characterization of the Parent and the Ts-ORT Strains

The wild-type parent strain of ORT and the Ts-ORT strain were grown as previously described at 41° C. and 31° C., respectively. Wild-type and Ts-ORT strains were gram-stained following standard procedures. Colonial morphology was evaluated on SBA plates after a 48-hr incubation, and bacterial morphology was observed using optical microscopy (1000x).

Biochemical and enzymatic identification of wild-type and Ts-ORT strains were performed using commercial test kits, API 20 E System and API ZYM (BioMerieux, Saint Louis, Mo.) following manufacturer's instructions.

### Example 7

### Random Amplified Polymorphic DNA Fingerprinting

A polymerase chain reaction (PCR)-based technique was performed for DNA fingerprinting as described (Chiu et al., 2000, Emerg. Infect. Dis., 6:481-6). Briefly, bacterial strains were grown in SBA plates under microaerophilic conditions as described herein. The Ts-ORT strain was incubated at 31° C. for 48 hrs, and the wild-type strain at 41° C. for 24 hrs. Bacterial cells were harvested and homogenated with 250 μl of Tween 20 and TE buffer, and then incubated at 94° C. for 20 mins. The suspension was centrifuged at 17,530×g for 4 mins after addition of 250 μl of chloroform. The supernatant was collected, and the DNA was quantified by ultraviolet absorbance at A₂₆₀. The arbitrary 10-mer primers (Genosys, Woodlands, Tex.) used for this PCR-based fingerprinting method were:

5′-GTGCAATGAG-3′ (SEQ ID NO:1);

5′-GTGCAATGAG -3′ (SEQ ID NO:2);

5′-GTGCAATGAG -3′ (SEQ ID NO:3);

5′-GTGCAATGAG -3′ (SEQ ID NO:4);

5′-GTGCAATGAG-3′ (SEQ ID NO:5);

5′-GTGCAATGAG -3′ (SEQ ID NO:6);

5′-GTGCAATGAG -3′ (SEQ ID NO:7);

5′-GTGCAATGAG-3′ (SEQ ID NO:8);

5′-GTGCAATGAG -3′ (SEQ ID NO:9); and

5′-GTGCAATGAG-3′ (SEQ ID NO:10).

Each random amplified polymorphic DNA (RAPD) reaction was performed containing 15 ng of genomic DNA, 10 mM of oligonucleotides, 1 unit of Taq Polymerase, 10 mM Tris-HCl, 1.5 mM MgCl₂, and 50 mM KCl pH 8.3 (all reagents provided by Roche Diagnostics Corp., Indianapolis, Ind.). The amplification was performed in Perkin Elmer Thermal Cycler (PE Biosystems, Foster City, Calif.) at 95° C. for 5 mins for initial denaturation, followed by 35 cycles of denaturation (94° C. for 30 sees), annealing (42° C. for 2 mins) and extension (72° C. for 2 mins). The final extension was executed at 72° C. for 7 mins. The final products of the PCR were separated using a 2% agarose gel, and then visualized and photographed under ultraviolet light.

### Example 8

### Repetitive Polymerase Chain Reaction

PCR-based fingerprinting was performed using repetitive extragenic palandromic (REP), enterobacterial repetitive intergenic consensus (ERIC), Salmonella enteritidis repetitive element (SERE), and BOX primers that are described elsewhere (Alam et al., 1999, J. Clin. Microb., 37:2772-6; Rajashekara et al., 1998, J. Med. Microbiol., 47:489-98; Versalovic & Lupski, 1991, Nuc. Acids Res., 19:6823-31). Bacterial strains were grown in SBA plates under microaerophilic conditions. The Ts-ORT strain was incubated at 31° C. for 48 hrs, and the wild-type strain at 41° C. for 24 hrs. Bacterial cells were harvested, and the DNA was extracted and quantified as described herein. The oligonucleotide primers (Genosys) used were:

ERIC1R (5′-ATG TAA GCT CCT GGG GAT TCA C-3′) (SEQ ID NO:11),

ERIC2 (5′-AAG TAA GTG ACT GGG GTG AGC G-3′) (SEQ ID NO:12),

REP1R (5′-IIII CGI CGI CAT CIG GC-3′) (SEQ ID NO:13),

REP2 (5′-ICG ICT TAT CIG GCC TAC-3′) (SEQ ID NO:14),

SERE (5′ GTG AGT ATA TTA GCA TCC GCA -3′) (SEQ ID NO:15), and

BOX (5′ ATA CTC TTC GAA AAT CTC TTC AAA C-3′) (SEQ ID NO:16).

Each rep-PCR reaction was performed containing 15 ng of genomic DNA, 10 mM of oligonucleotides, 1 unit of Taq polymerase, 10 mM Tris-HCl, 1.5 mM MgCl₂, and 50 mM KCl pH 8.3 (all reagents were from Roche Diagnostics Corp.). All PCR-based reactions were performed in a Perkin Elmer Thermal Cycler (PE Biosystems, Foster City, Calif.). The amplifications using ERIC (ERIC1R and ERIC2), REP (REP1R and REP2), and SERE primers were initiated at 95° C. for 5 mins for initial denaturation, followed by 35 cycles of denaturation (94° C. for 30 secs), annealing (40° C. for 2 mins), and extension (72° C. for 2 mins). The final extension was executed at 72° C. for 7 mins. Rep-PCR based on BOX primers was performed at 95° C. for 5 mins for initial denaturation, followed by 35 cycles of denaturation (94° C. for 30 secs), annealing (52° C. for 2 mins), extension (72° C. for 2 mins) and final extension at 72° C. for 7 mins. The final PCR products were separated using a 2% agarose gel, and then visualized and photographed under UV light.

The VL mORT 108a-10.6 Ts-ORT strain was characterized according to colony and cellular morphology. The Ts-ORT strain exhibited slower growth and smaller colony size (0.5-1.5 mm) when compared to the wild-type ORT strain (1 to 3 mm). Colonial and cellular morphologies of the Ts-ORT were found to be similar to the wild-type strain. There were no differences in biochemical and enzymatic reactions tested in wild-type and Ts-ORT strains. Strains were also characterized using a PCR-based fingerprinting method. The results revealed similar patterns of amplification products representing repetitive sequences (ERIC, BOX, REP and SERE), however, the random amplified polymorphic DNA (RAPD) fingerprinting was able to differentiate Ts-ORT and wild-type strains showing a unique pattern.

### Example 9

### Colonization by the Ts-ORT Strain and the Serological Response Produced by Birds

One-hundred-eighty one day-old turkeys known to be free of ORT infection by ELISA and SPAT were equally divided in 6 groups and kept in isolation during the experiment. Two groups were administered one of two different concentrations (10⁷ and 10⁸ CFU/ml) of Ts-ORT strain in a three-hour supply of drinking water. Three other groups were instilled with 50 μl of one of the three different concentrations (10⁷, 10⁸ and 10⁹ CFU/ml) of Ts-ORT strain in each nostril and conjunctival space. Birds in one group were kept as controls. Blood samples were weekly collected from all groups.

The colonization of the upper respiratory tract by Ts-ORT strain in 1 day-old turkeys was assessed by culturing choanal and tracheal swabs every 3 days for 15 days. The swabs were examined by streaking them onto duplicate SBA plates that were incubated at either 31° C. (permissive temperature) or 41° C. (non-permissive) under microaerophilic conditions. The re-isolation of Ts-ORT strain was attempted, and the identities of the isolates were confirmed by biochemical tests.

Blood samples were collected from all groups at 0, 1, 2, and 3 weeks post-vaccination. The specific serological responses elicited to ORT were detected by OMP-ELISA as described herein.

Colonization of a Ts-ORT strain in the upper respiratory tract was studied in one-day old poults after administering different concentrations in the drinking water and by oculo-nasal instillation. Choanal and tracheal swabs were cultured at permissive and non-permissive temperatures. The Ts- ORT strain was recovered from swabs from all groups of birds incubated at 31° C., but not at 41° C., except non-administered controls, independent of the concentration of the Ts-ORT strain or the route of administration 13 days post-administration (Table 1). Birds in all groups were clinically normal, showing no signs of disease after administration of the Ts-ORT strain. Humoral immune response to ORT was detected by OMP-ELISA in 19% of treated birds, except controls, at three weeks post-administration independent of the concentration of Ts-ORT strain or the route of administration.

TABLE 1
Colonization of upper respiratory tract by the Ts-ORT strain in
turkeys by swab sampling of tracheas and choanae

| | Non- | Drinking water | | Oculonasal | | |
|---|---|---|---|---|---|---|
| | vaccinated | vaccination | | vaccination | | |
| Days | Control¹ | 10^{7♦} | 10^{8♦} | 10^{7♦} | 10^{8♦} | 10^{9♦} |
| 0 | 0/30* | 0/30 | 0/30 | 0/3 | 0/30 | 0/30 |
| 3 | 0/30 | 14/30 | 2/30 | 11/27 | 6/30 | 13/30 |
| 6 | 0/30 | 2/30 | 6/30 | 1/26 | 3/30 | 8/30 |
| 9 | 0/30 | 1/29 | 5/29 | 6/29 | 2/29 | 5/21 |
| 13 | 0/29 | 1/28 | 5/26 | 8/19 | 5/25 | 3/19 |
| 15 | 0/30 | 0/30 | 0/30 | 0/30 | 0/30 | 0/30 |

¹control group was kept as non-administered control
^{♦}number of CFU/ml administered to the group
*number of birds positive for Ts-ORT strain of ORT/total number of birds from swabs incubated at 31° C.

### Example 10

### Laboratory Evaluation of Ts-ORT Strain of ORT

At four weeks of age, turkeys from groups administered 10⁷ or 10⁸ of the Ts-ORT strain in drinking water or 10⁷, 10⁸ or 10⁹ of the Ts-ORT strain oculonasally and one group of non-vaccinated control were challenged with 1 ml of pathogenic ORT strain (10⁹ CFU/ml) via intratracheal route as previously described (Sprenger et al., 1998, Avian Dis., 42:154-61). Twenty blood samples collected randomly from each group were examined weekly for the presence of specific serological responses to ORT by OMP-ELISA following the procedure described herein. Seven turkeys were euthanized by carbon dioxide inhalation at 3, 7, and 9 days post-challenge. Clinical signs and gross lesions were evaluated according to a scoring system.

Lesions were evaluated according to their pathological significance by giving scores to each individual of the group as follows: “none” (0), “tracheitis” (1), “liver congestion” (1), “airsacculitis” (2) and “lung consolidation” (3). Each lesion in internal organs was also evaluated according to severity as “mild” (1), “moderate” (2) and “severe” (3). The lesion site and severity scores were individually recorded for each individual of each group. Final scores for each individual of a group were given by multiplying the lesion site score by the severity score. The mean score of gross lesions of each group was calculated as the sum of all individual final scores divided by the number of individuals in the given group.

Protection against a challenge was measured in mean scores of gross lesions for individual groups. Results from each group were compared by Poisson regression assuming a Poisson distribution. Values of p<0.05 were considered significant. Statistical analyses tested (i) whether the 5 different concentrations of the treatment were significantly different from each other, and (ii) whether the control was significantly different from the treatment.

### Example 11

### Field Evaluation of Ts-ORT Strain

Ts-ORT strain was grown in SBA plates at 31° C. in conditions as described herein. The bacterial cells were aseptically harvested and initially diluted in sterile PBS (pH 7.4).

Forty-six-thousand one-day-old commercial turkeys were equally divided into two adjacent houses in a commercial turkey farm. Twenty randomly selected serum samples were examined for ORT antibodies to ensure absence of specific antibodies to ORT before vaccination by ELISA and SPAT as described herein. The Ts-ORT strain was administered to 5 day-old poults in the drinking water through a conventional watering system to 23,000 poults. Prior to mixing the vaccine, the water system was cleaned and made free of all medications and disinfectants. Drinking water was buffered with powdered milk (1 lb/100 gal of drinking water), and Ts-ORT was added to the water tank containing 3 to 4 hours of drinking water supply to a final concentration of 10⁶ CFU/ml. One group of 23,000 poults was kept as non-vaccinated controls in an adjacent turkey house. Twenty blood samples from both vaccinated and non-vaccinated groups were tested for specific serological response to ORT by OMP-ELISA at weekly intervals. Serum samples were analyzed in 1:2 dilutions and expressed in Geometric Mean Titers (GMT). At 7 wks post-vaccination, 35 turkeys from the vaccinated group and 35 turkeys from the non-vaccinated control group were transferred to isolation. Twenty-five non-vaccinated and 25 vaccinated turkeys were challenged with 1 ml of pathogenic ORT strain (7×10⁸ CFU/ml) via intratracheal route as described previously (Sprenger et al., 1998). Ten non-vaccinated turkeys were kept as a non-challenged control group. One group containing 10 vaccinated turkeys was kept as a vaccinated/non-challenged group. Eight turkeys in all groups were euthanized by carbon dioxide inhalation at 3, 7, and 10 days post-challenge. Clinical signs and gross lesions were evaluated according to the scoring system previously described.

Swabs from lung, air sacs, and trachea were collected during necropsy from each individual of all groups and cultured on SBA at 41° C. under microaerophilic conditions for at least 48 hrs. Suspicious colonies were subcultured, and their identities were confirmed by biochemical testing.

Samples of lung tissue from each individual of each group were aseptically collected in sterile Whirl-Pak bags (Nasco Inc., Fort Atkinson, Wis.) during necropsy and kept under refrigeration until laboratory processing. Tissues were individually homogenated in sterile TSB in a dilution of 1 gram of lung tissue per milliliter of TSB. Homogenate was plated on SBA plates that were then cultured at 41° C. under microaerophilic conditions for at least 48 hrs. Suspicious colonies were quantified and subcultured, and the identities were confirmed by biochemical testing and agglutination of bacterial cells with ORT hyperimmune serum. Final quantification of ORT was determined in CFU per gram of lung tissue.

Sections of lung, liver, trachea, and spleen were collected at necropsy and fixed in 10% PBS formaldehyde solution. Tissues were embedded in paraffin, sectioned in 4 μm, and stained with hematoxylin and eosin according to standard procedures (Sheehan & Hrapchak, 1987, Theory and Practice of Histotechnology, 2^{nd} Ed., Battele Mem. Inst., Battele Press, Columbus, Ohio). Slides containing tissues were examined and photographed using bifocal optical microscope (Nikon Inc., Japan) at 200× and 400× amplification.

Protection against challenge in all groups was compared based on the mean score of gross lesions, re-isolation of ORT, and a quantification of CFU of pathogenic strain of ORT per gram of pulmonary tissue. Results of mean scores of gross lesions and quantification of CFU per gram of lung tissue in vaccinated and non-vaccinated groups were analyzed by Poisson regression assuming a Poisson distribution. Re-isolation rates of ORT of different tissues from vaccinated, non-vaccinated, and controls (vaccinated and non-vaccinated controls) were compared using a Chi-Squared test on the data. Values of p<0.05 were considered significant.

### Example 12

### Evaluation of the Serological Response of Laboratory Turkeys to the Ts-ORT Strain

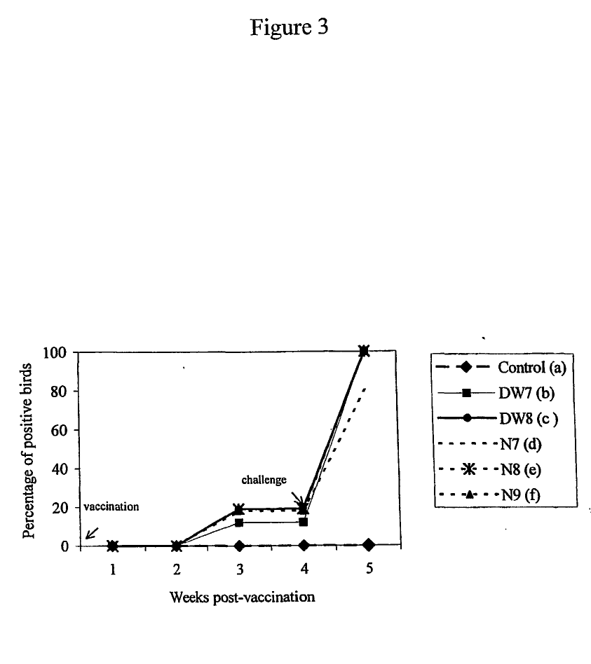

Blood samples were collected from all groups of birds for 4 wks after administration of the Ts-ORT strain and 7 days after inoculation of a pathogenic strain of ORT. Serum samples were tested for ORT specific antibodies by OMP-ELISA. In all treated groups, 12-19% of birds seroconverted to ORT by OMP-ELISA at 3 wks post-administration independent of concentration of Ts-ORT strain or administration route, but not control group. At 7 days post-inoculation of the pathogenic strain, all birds that were given the Ts-ORT strain were positive for antibodies for ORT. Birds in control group did not seroconvert until 7 days post-inoculation. The results showed in FIG. 3 are expressed as a percentage of positive birds for ORT antibodies.

Protection against a pathogenic strain of ORT was assessed by scoring gross lesions in birds in control and treated groups. Birds in the non-vaccinated/challenged control group reached 5 in the scoring scale and presented bloody nasal discharge for up to 5 days post-challenge. Birds in groups that were administered the Ts-ORT strain by drinking water or oculonasal instillation in different concentrations showed scores varying from 1 to 2. Statistically, by Poisson regression, the results indicated no significant differences among different concentrations of the treatment (p value=0.31). However, the difference between the 5 treatments and control was considered highly significant (p value=0.00). Table 2 presents results of gross lesion scores in vaccinated and non-vaccinated turkeys after challenge with a pathogenic strain of ORT.

TABLE 2
Mean scores of gross lesions in turkeys vaccinated and non-vaccinated
with Ts-strain after challenge with pathogenic strain of ORT

| | Non- | Drinking water | | Oculo-nasal | | |
|---|---|---|---|---|---|---|
| | vaccinated | vaccination | | vaccination | | |
| Days | Control | 10⁷* | 10⁸* | 10⁷* | 10⁸* | 10⁹* |
| 3 | 5¹ | 1¹ | 2¹ | 2¹ | 1¹ | 1¹ |
| 6 | 3 | 0 | 0 | 0 | 0 | 0 |
| 9 | 0 | 0 | 0 | 0 | 0 | 0 |

*number of CFU/ml of Ts-ORT strain given to birds
¹mean score of gross lesions in turkeys after challenge

### Example 13

### Evaluation of the Serological Response of Field Turkeys to the Ts-ORT Strain

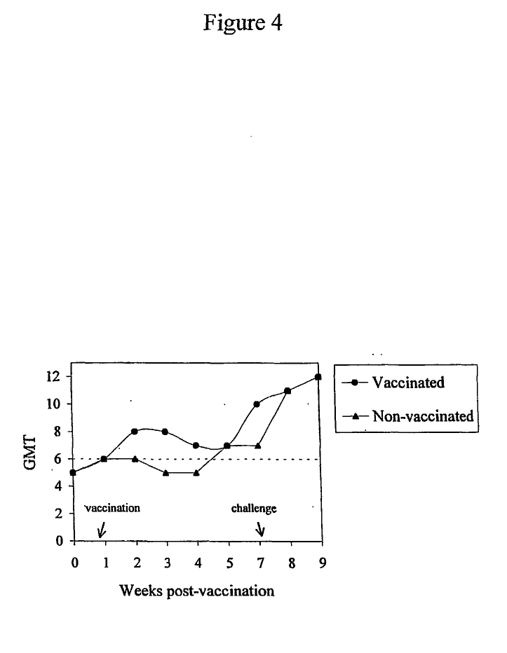

Turkeys in the vaccinated group developed antibody titers for ORT after 1 wk post-vaccination with the Ts-ORT strain in the drinking water under field conditions. Titers above GMT 6 were considered positive for ORT antibodies based on negative control sera. After challenge with a pathogenic strain of ORT, both vaccinated and non-vaccinated groups exhibited an increase in GMT titers, except for the non-vaccinated/non-challenged group. FIG. 4 shows serological responses in the vaccinated and non-vaccinated groups expressed in GMT titers.

Gross lesions in vaccinated and non-vaccinated groups were evaluated using the described scoring system. Non-vaccinated/challenged turkeys presented severe airsacculitis and moderate-severe lung lesions at 3, 6, and 10 days post-challenge when compared to vaccinated/challenged turkeys that had mild airsacculitis at 3 days post-challenge only. Table 3 shows mean scores of gross lesion for vaccinated and non-vaccinated groups after challenge with pathogenic strain. Birds in the non-vaccinated/challenged group exhibited higher mean scores for gross lesions (5.0-6.5) at 3, 6, and 10 days post-inoculation when compared to the vaccinated/challenged group. Vaccinated/challenged birds showed a mean score from 1 to 2. Vaccinated/non-challenged group had no scoring at 3, 6, and 10 days post-inoculation. Non-vaccinated/non-challenged group had a score interval of 3.5-4. By Poisson regression test, the difference between the mean scores of gross lesions in vaccinated/challenged and non-vaccinated/challenged groups were highly significant (p value=0.00). The mean scores of gross lesions for the non-vaccinated/challenged group were calculated to be from 3 to 7 times the mean number of lesions for the vaccinated/challenged group considering a 95% confidence interval.

The vaccinated/challenged group exhibited significantly lower re-isolation ratios (p value=0.0000278) from trachea, air sacs, and lungs when compared to the non-vaccinated groups (Table 4). Quantification of CFU/gram of lung tissue was also significantly lower (p value=0.0020) in the vaccinated/challenged group (2.5-4.4 CFU/g) as compared to the non-vaccinated/challenged group (34.6-200.0 CFU/g) (Table 5).

Microscopically, the lungs of both vaccinated/challenged and non-vaccinated/non-challenged were hyperemic. The parabronchial lumen from non-vaccinated/challenged turkey was filled with suppurative exudate and surrounded by infiltrations of macrophages and multinucleated giant cells. The tracheal mucosa of either vaccinated or non-vaccinated turkeys had an infiltration with lymphocytes, plasma cells, and macrophages. No significant changes were detected in spleens and livers of vaccinated and non-vaccinated groups.

TABLE 3
Mean scores of gross lesions after challenge with pathogenic strain of
ORT in vaccinated and non-vaccinated groups in field study

| | Vaccinated/ | | | Non- |
|---|---|---|---|---|
| Days after | non- | Non-vaccinated/ | Vaccinated/ | vaccinated/ |
| challenge | challenged | non-challenged | challenged | challenged |
| 3 | 0 | 4 | 1 | 5.0 |
| 6 | 0 | 4 | 2 | 6.5 |
| 10 | 0 | 3.5 | 1 | 5.0 |

TABLE 4
Re-isolation of ORT in vaccinated, non-vaccinated and control groups of
turkeys following challenge with a pathogenic strain of ORT

| Days after | Site of | Vaccinated/ | Non-vaccinated/ | Vaccinated/ | Non-vacci [symbol] |
|---|---|---|---|---|---|
| challenge | re-isolation | non-challenged | non-challenged | challenged | challen [symbol] |
| 3 | trachea | 0/3¹ (0.0)² | 0/3¹ (0.0)² | 1/8¹ (12.5)² | 0/8¹ (0. [symbol] |
| | air sacs | 0/3 (0.0) | 0/3 (0.0) | 1/8 (12.5) | 2/8 (25 [symbol] |
| | lungs | 0/3 (0.0) | 0/3 (0.0) | 1/8 (12.5) | 5/8 (62 [symbol] |
| 6 | trachea | 0/3 (0.0) | 0/3 (0.0) | 1/8 (12.5) | 0/8 (0. [symbol] |
| | air sacs | 0/3 (0.0) | 0/3 (0.0) | 0/8 (0.0) | 1/8 (12 [symbol] |
| | lungs | 0/3 (0.0) | 0/3 (0.0) | 3/8 (37.5) | 4/8 (50 [symbol] |
| 10 | trachea | 0/4 (0.0) | 0/4 (0.0) | 1/9 (11.1) | 3/9 (33 [symbol] |
| | air sacs | 0/4 (0.0) | 0/4 (0.0) | 1/9 (11.1) | 2/9 (22 [symbol] |
| | lungs | 0/4 (0.0) | 0/4 (0.0) | 1/9 (11.1) | 5/9 (55 [symbol] |

¹number of turkeys with ORT re-isolations/total number of turkeys
²percentage of birds with ORT re-isolation

TABLE 5
Quantification of colony forming units of ORT wild-type strain in
pulmonary tissues in control groups, vaccinated and non-vaccinated
turkeys after challenge with a pathogenic strain of ORT

| | Vaccinated/ | | | Non- |
|---|---|---|---|---|
| Days after | non- | Non-vaccinated/ | Vaccinated/ | vaccinated/ |
| challenge | challenged | non-challenged | challenged | challenged |
| 3 | 0* | 2.5* | 3* | 200.0* |
| 6 | 0 | 4.4 | 6 | 53.6 |
| 10 | 0 | 3.3 | 10 | 34.6 |

*results are given in average of CFU per gram of pulmonary tissue in the given group of turkeys

### Example 14

### ORT in Egg Laying Hens

The following study was conducted using egg production hens. Briefly, 20 serum samples were collected per flock. The sample set included 52 flocks ranging in age from 1 day to 304 weeks old. There were a total of 925 samples analyzed for the presence of antibodies against ORT using the SPAT and OMP-ELISA following the procedure described herein. The results from the samples were recorded and grouped in one of two age groups. The prevalence was calculated for each age group considering results by total and individual states.

The results for each age group and serologic test are presented in Tables 6 and 7. None of the one-day old chicks were found positive by either test. Both age groups had a higher prevalence of ORT by ELISA than by the SPAT test. For flocks less than 22 weeks of age, 310 serum samples were collected of which 82 were positive using the ELISA test and 30 were positive using the SPAT test. Of the 615 serum samples collected from birds at 22 weeks of age and older, 424 and 164 samples were positive for the presence of antibodies to ORT by ELISA and SPAT, respectively.

Results demonstrated that layer flocks had greater serologic prevalence for ORT than pullets. All layer flocks, but only 52% of pullet flocks, were positive by ELISA. The consistently higher prevalence of antibodies found by the ELISA compared to the SPAT results can be explained by the more sensitive nature of the ELISA test. The SPAT is most sensitive within the first two weeks following an infection. The sensitivity of the ELISA lasts for at least eight weeks post infection.

TABLE 6
Serological incidence of ORT antibodies in commercial laying chickens in four Midwestern states

| Origin | | Number | Number | Number of samples | | Number of flocks | |
|---|---|---|---|---|---|---|---|
| of | Age of birds at | of flocks | of samples | positive for ORT | | positive for ORT | |
| samples | sampling (weeks) | tested | tested | SPAT | ELISA | SPAT | ELISA |
| MN¹ | Less than 22 weeks | 6 | 111 | 14 (13)⁶ | 41 (37)⁶ | 4 (67)⁶ | 4 (67)⁶ |
| MN | 22 weeks and older | 16 | 318 | 106 (33) | 241 (76) | 15 (94) | 16 (100) |
| NE² | Less than 22 weeks | 4 | 58 | 5 (9) | 26 (45) | 3 (75) | 4 (100) |
| NE | 22 weeks and older | 7 | 140 | 26 (19) | 86 (61) | 6 (86) | 7 (100) |
| WI³ | Less than 22 weeks | 1 | 14 | 3 (21) | 5 (36) | 1 (100) | 1 (100) |
| WI | 22 weeks and older | 2 | 37 | 20 (54) | 35 (95) | 2 (100) | 2 (100) |
| IA⁴ | Less than 22 weeks | 10 | 127 | 8 (6) | 10 (8) | 1 (10) | 2 (20) |
| IA | 22 weeks and older | 6 | 120 | 12 (10) | 62 (52) | 5 (83) | 6 (100) |
| Midw. | Less than 22 weeks | 21 | 310 | 30 (10) | 82 (26) | 9 (43) | 11 (52) |
| states⁵ | | | | | | | |
| Midw. | 22 weeks and older | 31 | 615 | 164 (27) | 424 (69) | 28 (90) | 31 (100) |
| states | | | | | | | |

¹Minnesota.
²Nebraska.
³Wisconsin.
⁴Iowa.
⁵Midwest states (Minnesota, Nebraska, Wisconsin and Iowa)
⁶results are given in percentage of positive results.

TABLE 7
Serological incidence of ORT antibodies in commercial
laying chickens for all age groups.

| | | Num- | | | | |
|---|---|---|---|---|---|---|
| | Number | ber of | | | | |
| Origin | of | sam- | Number of samples | | Number of flocks | |
| of | flocks | ples | positive for ORT | | positive for ORT | |
| samples | tested | tested | SPAT | ELISA | SPAT | ELISA |
| MN¹ | 22 | 429 | 120 (28)⁶ | 282 (66)⁶ | 19 (86)⁶ | 20 (90)⁶ |
| NE² | 11 | 198 | 31 (16) | 112 (57) | 9 (82) | 11 (100) |
| WI³ | 3 | 51 | 23 (45) | 40 (78) | 3 (100) | 3 (100) |
| IA⁴ | 16 | 247 | 20 (8) | 72 (29) | 6 (37) | 8 (50) |
| Midw. | 52 | 925 | 194 (21) | 506 (55) | 37 (71) | 42 (81) |
| states⁵ | | | | | | |

¹Minnesota.
²Nebraska
³Wisconsin.
⁴Iowa.
⁵Midwest states (Minnesota, Nebraska, Wisconsin and Iowa)
⁶results are given in percentage of positive results.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising an isolated mutant ORT organism.

2. The composition of claim 1, wherein said isolated mutant ORT organism is attenuated.

3. The composition of claim 1, wherein said isolated mutant ORT organism is a temperature sensitive mutant ORT organism having a permissive temperature for growth and a non-permissive temperature for growth.

4. The composition of claim 3, wherein said permissive temperature comprises 31° C.

5. The composition of claim 4, wherein said permissive temperature is from about 29° C. to about 33° C.

6. The composition of claim 3, wherein said non-permissive temperature comprises 41° C.

7. The composition of claim 6, wherein said non-permissive temperature comprises below 29° C. and above 33° C.

8. The composition of claim 3, wherein said mutant temperature sensitive ORT organism is alive.

9. The composition of claim 3, wherein said mutant temperature sensitive ORT organism comprises the distinguishing characteristics of the organism assigned ATCC Accession number ______.

10. The composition of claim 3, wherein said mutant temperature sensitive ORT organism is VL mORT 108a-10.6.

11. The composition of claim 1, wherein said composition, after administration, protects a bird from an ORT infection.

12. The composition of claim 1, wherein said composition is effective for lowering the risk of an ORT infection in a bird.

13. A vaccine comprising an effective amount of a mutant ORT organism.

14. The vaccine of claim 13, wherein said isolated mutant ORT organism is attenuated.

15. The vaccine of claim 13, wherein said isolated mutant ORT organism is a temperature sensitive mutant ORT organism having a permissive temperature for growth and a non-permissive temperature for growth.

16. The vaccine of claim 15, wherein said permissive temperature comprises 31° C.

17. The vaccine of claim 16, wherein said permissive temperature is from about 29° C. to about 33° C.

18. The vaccine of claim 15, wherein said non-permissive temperature comprises 41° C.

19. The vaccine of claim 18, wherein said non-permissive temperature comprises below 29° C. and above 33° C.

20. The vaccine of claim 13, wherein said mutant temperature sensitive ORT organism comprises the distinguishing characteristics of the organism assigned ATCC Accession number ______.

21. The vaccine of claim 13, wherein said mutant temperature sensitive ORT organism is VL mORT 108a-10.6.

22. A method for protecting a bird from an ORT infection, said method comprising administering to said bird a composition comprising an effective amount of an isolated mutant ORT organism.

23. A method for reducing the risk of an ORT infection in a bird, comprising administering to said bird a composition comprising an isolated mutant ORT organism.

24. The method of claim 22, wherein said composition is applied to an eye or a nostril of said bird.

25. The method of claim 22, wherein said composition is supplied in the drinking water of said bird.

26. The method of claim 22, wherein said bird is a turkey or a chicken.

27. The method of claim 22, wherein said isolated mutant ORT organism is attenuated.

28. The method of claim 22, wherein said isolated mutant ORT organism is a temperature sensitive mutant ORT organism having a permissive temperature and a non-permissive temperature for growth.

29. The method of claim 28, wherein said permissive temperature for growth comprises 31° C.

30. The method of claim 28, wherein said non-permissive temperature comprises 41° C.

31. An inoculated bird, wherein said bird comprises anti-ORT antibodies due to inoculation with an isolated mutant ORT organism.

32. The bird of claim 31, wherein said bird is a turkey or a chicken.

33. A body part of said bird of claim 32.

34. The body part of claim 33, wherein said body part comprises a meat portion.

35. A method for identifying a bird that is or was infected with ORT, said method comprising:
a) contacting a biological sample from said bird with an ORT antigen under conditions wherein said ORT antigen specifically binds to an anti-ORT antibody, if present in said biological sample, to form an antibody-antigen complex; and
b) detecting the presence or absence of said antibody-antigen complex, the presence of said antibody-antigen complex indicating that said bird is or was infected with ORT.

36. The method of claim 35, wherein said bird is a turkey or a chicken.

37. The method of claim 35, wherein said ORT antigen comprises an ORT OMP preparation.

38. The method of claim 37, wherein said ORT OMP preparation is an ORT serotype A OMP preparation, an ORT serotype C OMP preparation, an ORT serotype E OMP preparation, or an ORT serotype I OMP preparation.

39. The method of claim 35, wherein said ORT antigen comprises a 70% pure ORT polypeptide.

40. The method of claim 35, wherein said ORT antigen is immobilized on a solid support.

41. The method of claim 40, wherein said solid support is selected from the group consisting of a dipstick, a microtiter plate, a bead, an affinity column, and an immunoblot membrane.

42. The method of claim 35, wherein said biological sample comprises serum.

43. The method of claim 35, wherein said detecting step comprises performing an enzyme-linked immunoassay, a radioimmunoassay, an immunoprecipitation, or an immunoblot assay.

44. The method of claim 35, wherein said detecting step comprises: contacting said antibody-antigen complex with an indicator molecule that selectively binds to said anti-ORT antibody; and detecting the presence of said indicator molecule.

45. A method for detecting an ORT infection in a bird, said method comprising:
a) contacting a biological sample from said bird with an anti-ORT antibody under conditions wherein said anti-ORT antibody specifically binds to an ORT antigen, if present in said biological sample, to form an antibody-antigen complex; and
b) determining the presence or absence of said antibody-antigen complex, the presence of said antibody-antigen complex indicating that said bird has said infection.

46. The method of claim 45, wherein said bird is a turkey or a chicken.

47. The method of claim 45, wherein said ORT antigen comprises a portion of an ORT organism.

48. The method of claim 45, wherein said ORT antigen is one or more ORT OMPs.

49. The method of claim 45, wherein said anti-ORT antibody is immobilized on a solid support.

50. The method of claim 45, wherein said anti-ORT antibody has specific binding affinity for an OMP polypeptide.

51. The method of claim 45, wherein said biological sample comprises a tracheal swab.

52. A composition comprising a substantially pure ORT antigen.

53. A composition consisting essentially of an ORT OMP preparation.

54. A composition comprising an ORT antigen and an indicator molecule.

55. The composition of claim 54, further comprising one or more antigens for detecting a plurality of avian infections.

56. The composition of claim 54, further comprising one or more antibodies for detecting a plurality of avian infections.

57. The composition of claim 55, wherein said plurality of avian infections is caused by an organism selected from the group consisting of Salmonella spp., Bordetella avian, avian pneumovirus, avian encephalitis virus, avian influenza, avian leukosis, fowl pox, infectious bronchitis virus, infectious bursal disease virus, Newcastle dsease virus and reovirus.

58. The composition of claim 54, wherein said indicator molecule selectively binds to anti-ORT antibodies produced by the bird species from which said biological sample is obtained.

59. The composition of claim 54, wherein said ORT antigen is immobilized on a solid substrate.

60. The composition of claim 59, wherein said solid substrate is selected from the group consisting of a dipstick, a microtiter plate, a bead, an affinity column, an immunoblot membrane, and an immunoblot paper.

61. A composition comprising an anti-ORT antibody and an indicator molecule.

62. The composition of claim 61, further comprising one or more antigens for detecting a plurality of avian infections.

63. The composition of claim 61, further comprising one or more antibodies for detecting a plurality of avian infections.

64. The composition of claim 63, wherein said plurality of avian infections is caused by an organism selected from the group consisting of Salmonella spp., Bordetella avian, avian pneumovirus, avian encephalitis virus, avian influenza, avian leukosis, fowl pox, infectious bronchitis virus, infectious bursal disease virus, Newcastle dsease virus and reovirus.

65. The composition of claim 61, wherein said indicator molecule selectively binds to anti-ORT antibodies produced by the bird species from which said biological sample is obtained.

66. The composition of claim 61, wherein said ORT antigen is immobilized on a solid substrate.

67. The composition of claim 66, wherein said solid substrate is selected from the group consisting of a dipstick, a microtiter plate, a bead, an affinity column, an immunoblot membrane, and an immunoblot paper.
